(19) United States
(12) Patent Application Publication
(10) Pub. No.: US 2010/0227307 A1
(43) Pub. Date: Sep. 9, 2010

(54) ERGOTHIONEINE AND/OR ITS DERIVATIVES AS A CELL PRESERVATIVE

(76) Inventors: Marvin S. Hausman, Stevenson, WA (US); Elizabeth S. Metcalf, Sherwood, OR (US)

Correspondence Address: KLAUBER & JACKSON, 411 HACKENSACK AVENUE, HACKENSACK, NJ, 07601, US

(21) Appl. No.: 12/595,506

(22) PCT Filed: Apr. 10, 2008
(86) PCT No.: PCT/US08/59831

§ 371 Date: May 18, 2010

Related U.S. Application Data
(60) Provisional application No. 60/911,391, filed on Apr. 12, 2007.

(51) Int. Cl. A01N 1/02; C12N 5/071; C12N 5/04; C12N 1/20; C12N 5/07

(52) U.S. Cl. 435/2; 435/374; 435/420; 435/252.1; 435/348

(57) ABSTRACT

The present invention relates to cell, tissue or derivatives thereof, preserving compositions for cells in culture, storage, or lyophilization. According to the invention ergothioneine is added as a supplement to such extender or preservative compositions. Sperm cells so treated showed increased motility when thawed compared to cells that were not treated with ergothioneine.

### CROSS REFERENCE TO RELATED APPLICATIONS

The present application is a National Stage Application claiming the priority of co-pending PCT Application No. PCT/US2008/059831 filed Apr. 10, 2008, which in turn, claims priority from U.S. Provisional application Ser. No. 60/911,391 filed Apr. 12, 2007. Applicants claim the benefits of 35 U.S.C. §120 as to the PCT application and priority under 35 U.S.C. §119 as to the said United States provisional application, and the entire disclosures of both applications are incorporated herein by reference in their entireties.

### BACKGROUND OF THE INVENTION

1. Field of the Invention

The present invention relates to a media for preserving animal or plant cells in suspension, in culture for short or long term storage as well as a novel preservative compositions containing the same.

2. Background Art

Long term storage of plant and animal cells, their derivatives and tissues is of widespread critical importance to the research and biomedical fields. Cryopreservation of cells is useful for the long-term storage of cell lines to provide unchanging populations of cells; and the storage of cells for research or medical purposes.

Many cells used in biomedical research are currently stored and transported in a cryopreserved state in a liquid nitrogen bath. When researchers thaw the cells for use in the lab, however, less than 1% remain viable. The few surviving cells must be placed in culture and painstakingly tended to for weeks before new colonies are abundant enough to be useful for experiments or therapy. The low survival rate makes working with the stem cells time and labour intensive. Furthermore because so few cells survive freezing, natural selection may be altering cell lines in unknown and undesired ways.

Animal cells can be stored indefinitely once they reach liquid nitrogen temperature (−196.degree. C.). It has been well-established, however, that the freezing process itself results in immediate and long-term damage to cells with the greatest damage occurring to cells as they traverse the intermediate zone of temperature (−15.degree. C. to −60.degree. C.) during cooling and thawing (Mazur, Am. J. Physiol., 247:C125-142, 1984). The primary damaging physical events that can occur to cells during the process of freezing include dehydration and intracellular ice crystal formation. During freezing, solute is rejected from the solid phase producing an abrupt change in concentration in the unfrozen portion of solution. A biological cell responds to this perturbation by dehydrating to reach a new equilibrium state between intracellular and extracellular solutions. At high cooling rates, equilibrium cannot be maintained because the rate at which the chemical potential in the extracellular solution is being lowered is much greater than the rate at which water can diffuse out of the cell. The end result of this imbalance is that intracellular ice formation is observed which is lethal to the cell (Toner, J. of Applied Phys., 67:1582-1593, 1990). At low cooling rates, cells are exposed for long periods of time at high subzero temperatures to high extracellular concentrations resulting in potentially damaging high intracellular concentrations (Lovelock, Biochem. Biophys. Acta, 10:414-446, 1953).

There have been attempts in the art to incorporate the process of vitrification into methods of cryopreserving cells. The aim of vitrification is to lower the temperature of a cell suspension while avoiding the formation of ice crystals by the use of viscous or concentrated liquid solutions. This approach is fundamentally different to standard methods of freezing that concentrate more so on carefully controlling the formation of ice crystals Methods incorporating vitrification have shown some promise however recoveries can be poor. Furthermore, the methods are not amenable to automation, and therefore quality control can be difficult. Another problem is that compounds such as polyethylene glycol are required in the vitrification solution. A further problem with vitrification is that the vessels used severely limit the amount of material that can be frozen. Additionally, the commonly used “open straws” do little to avoid the possibility of microbial cross-contamination of the material to be frozen.

The clinical and commercial application of cryopreservation for certain cell types is limited by the ability to recover a significant number of total viable cells that function normally. Significant losses in cell viability are observed in certain primary cell types. Examples of freeze-thaw cellular trauma have been encountered with cryopreservation of hepatocytes (Borel-Rinkes et al., Cell Transplantation, 1:281-292, 1992) porcine corneas (Hagenah and Bohnke, 30:396-406, 1993), bone marrow (Charak et al., Bone Marrow Transplantation, 11:147-154, 1993), porcine aortic valves (Feng et al., Eur. J. Cardiothorac. Surg., 6:251-255, 1992) and human embryonic stem cells (hESCs; http://www.wicell.org/forresearchers, FAQs—Culturing Human ES Cells: FAQs 4 & 8; Reubinoff et al., Human Reprod, 16(10):2187-2194, 2001).

Cryopreservation protocols typically require the use of cryoprotective agents (“CPAs”) to achieve improved survival rates for animal cells. A variety of substances have been used or investigated as potential additives to enhance survival of cells in the freezing process. Other substances used include sugars, polymers, alcohols and proteins. CPAs can be divided roughly into two different categories; substances that permeate the cell membrane and impermeable substances. One mechanism of protection results from reduction in the net concentration of ionic solutes for a subzero temperature when a CPA is present. This colligative effect is true for all substances that act as a CPA (Fahy, Biophys. J. 32:837-850, 1980). The addition of a CPA however, changes the ionicity of the solution. Both tissues and intact organs can exhibit reduced cellular viability when exposed to sufficiently large step changes in external osmolarity produced by introduction of a freezing solution (Pegg, Cryobiology, 9:411-419, 1972). In addition, long term exposure to even low concentrations of certain CPAs at room temperature is potentially damaging (Fahy, Cryobiology, 27: 247-268, 1990).

Another media component routinely added to freezing media to reduce cell damage and death during freezing and thawing is serum. This additive, however, is highly complex and may add a number of factors (known and unknown), which may interfere with or alter cell function. Other non-permeating protective agents such as ethylene glycol, polyvinyl pyrrolidone (Klebe and Mancuso, In Vitro, 19:167-170, 1983) sucrose, and culture medium (Shier and Olsen, In Vitro Cell Dev. Biol., 31:336-337, 1995), have been studied for their effectiveness as cryoprotective agents for cells with variable results. U.S. Pat. No. 4,004,975 to Lionetti et al. discloses the cryopreservation of leukocytes from centrifuged blood in a solution of hydroxyethyl starch and dimethylsulfoxide. U.S. Pat. No. 5,071,741 to Brockbank and PCT WO 92/08347 to Cryolife, published May 29, 1992, disclose the use of algae-derived polysaccharides such as agarose and alginate in a cryoprotective cell medium. U.S. Pat. No. 5,405,742 to Taylor discloses a solution for use as a blood substitute and for preserving tissue that includes dextran.

Artificial insemination (AI), along with in vitro fertilization and embryo transplantation, afford enhanced reproduction in mammals, including livestock, and offer many advantages over direct mating. In the livestock breeding art, these techniques permit wider dissemination of desirable genetic features. Semen collected from a single male can be used to inseminate multiple females, thereby reducing the number of males required to maintain a population. Artificial insemination techniques permit greater control over breeding, which results in greater reproducibility and facilitates maintenance of large-scale operations.

Maintaining the viability of reproductive cells is an important aspect of artificial insemination and other techniques used in indirect breeding. The processing requirements for semen used in AI may vary according to the species of animal. Bovine insemination requires relatively low concentrations of semen, and a suitable sample may be rapidly frozen in a narrow diameter straw and stored for an extended period of time without adversely affecting the fertility of the sample. In contrast, porcine semen is not susceptible to this approach because greater numbers of sperm cells and larger volumes of semen or diluted semen are required to inseminate sows. Insemination using frozen boar semen has not been sufficiently satisfactory to justify widespread use of this technique. Boar semen is generally diluted or extended with a suitable storage medium and cooled to a temperature of about 17.degree. C. prior to transport. The culture medium serves to increase the total volume of the sample and provide nutrients to maintain the sperm cells. Significant loss of sperm cell vitality occurs after storing the semen for just a few days. Currently, the best medium generally maintains boar sperm cell viability for about five to seven days. The relatively short time that boar semen can be stored imposes considerable constraints on the distribution of boar semen for AI. Other animals, such as horses, produce sperm cells that also suffer from short-lived viability.

Artificial insemination, in vitro fertilization, and embryo transfer technology are also used in humans to aid in the conception process, and/or as a solution to various physiological problems relating to infertility. Clearly, maintaining the viability of reproductive cells for these uses is also very important.

Accordingly, there is a need for safe and relatively inexpensive cell preservative compounds which improve cell viability after cell culture, long term storage, cryopreservation, even short term suspensions and the like.

### BRIEF SUMMARY OF THE INVENTION

The present inventors have discovered that cells of interest can efficiently be preserved when the cells are placed in a cell suspension, culture or extender which includes or is supplemented with ergothioneine and/or its derivatives. The addition of ergothioneine has been shown to improve viability of stored cells, particularly semen.

Accordingly, it is an object of the present invention to provide a supplement for viable cell storage, culture, suspension and the like which can result in increased cell survival and cell preservation and, improved viability of stored cells. The cells which may be preserved according to the invention include chondrocytes, red blood cells, stem cells, white blood cells, synoviocytes, plant cells, insect cells, bacterial cells and in a preferred embodiment, reproductive cells such as spermatozoa and oocytes as well as zygotes.

The supplement for cell preservation according to the present invention comprises ergothioneine (also known as thiotane or thiotaine) or an ergothioneine derivative.

Furthermore, the composition for cell culture, storage or suspension according to the present invention comprises at least the abovementioned culture medium supplement and a basal culture medium composition.

According to another embodiment of the present invention, there is provided a method of storing and reconstituting cells, which comprises the steps of adding the abovementioned storage medium supplement to a cell storage medium, storing said cells, and ultimately reconstituting said cells.

According to yet still another embodiment of the present invention, there is provided a method of replicating a virus vector of interest, which comprises the steps of adding the abovementioned medium supplement to a cell culture medium, culturing cells infected with the virus vector using the resulting medium for growth, and recovering the virus vectors from said medium and/or said cells.

According to a preferred embodiment, there is provided a method for extending the life of sperm cells used in artificial insemination which comprises the steps of adding the abovementioned medium supplement to a semen extender composition, culturing said semen cells in a medium and recovering the same.

According to another embodiment of the present invention, there is provided use of ergothioneine (also known as thiotane or thiotaine) or derivatives, homologs and functional equivalents of the same for producing a cell preservative composition. As used herein the term “ergothioneine” shall be interpreted to include ergothioneine derivatives, homologs, optical isomers, variants and the like which retain the cell preserving activity of ergothioneine.

Further, according to the present invention, safety of culture products can be enhanced since the amount of a serum component such as bovine fetal serum and bovine calf serum can be reduced or its use can be eliminated in culturing cells. The medium supplement according to the present invention is advantageous in terms of preparation and handling since cell viability can be preserved by adding it to a medium.

### BRIEF DESCRIPTION OF THE DRAWINGS

FIG. 1 is a graph showing semen cell total motility and FPM on semen treated with DMSO and EquiPro at 0, 30 minutes and 1 hour.

FIG. 2 is a graph depicting cell motility and FPM on thawed semen from IKE for cells treated with EquiPro and EquiPro plus 1 mM ergothioneine at 0, 30 minutes and 1 hour post thaw. One can see that the EquiPro supplemented with ergothioneine had superior motility.

FIG. 3 is a bar graph comparing thawed sperm cells from IKE treated with INRA and with INRA and ergothioneine. Again, the ergothioneine showed superior motility, particularly right after thaw.

### DETAILED DESCRIPTION OF THE PREFERRED EMBODIMENT

In one embodiment the invention relates to sperm cells. Semen for artificial insemination is often preserved by cooling or cryopreservation (freezing in LN2). Freezing semen is an effective preservation method, but there is a problem with maintaining potency of the spermatozoa after thawing. Semen extender compositions are frequently used to preserve the viability of the sperm after thawing. According to the invention, when ergothioneine was added to traditional semen extender compositions, there was a significant improvement in sperm cell survival and in surviving cell motility.

Many semen extender compositions utilize egg yolk. See, for example, U.S. Pat. No. 6,130,034 to Aitken; U.S. Pat. No. 3,444,039 to Rajamannan; U.S. Pat. No. 3,718,740 to Hafs et al.; and U.S. Pat. No. 3,973,003 to Kolas. A commercial semen extender composition that utilizes raw egg yolk is available under the name Biladyl® from Minitube GmbH. In general, the egg yolk is added to the composition just prior to the addition of the semen. Egg yolk serves as an external cryoprotectant of the sperm plasma membrane.

### Medium Supplement

A medium supplement for culturing cells according to the present invention comprises ergothioneine or an ergothioneine derivative. According to a preferred embodiment of the present invention, the medium supplement is used as a cell preservation agent.

L-ergothioneine is a naturally occurring antioxidant that is very stable in the body. It is synthesized in fungi and microorganisms and present in both plants and animals. Animals are unable to synthesize L-ergothioneine and must obtain it from dietary sources. It is readily absorbed and is active in most mammalian tissues, concentrating especially in the liver, where it prevents certain types of free-radical-induced damage to cell membranes and organelles. For example, exogenous L-ergothioneine has been shown to prevent lipid peroxidation by toxic compounds in the liver tissue of rats. Akanmu, D., et al., The antioxidant action of ergothioneine, Arch. of Biochemistry and Biophysics, 288 (1), 1991, pp. 10-16; Kawano, H., et al., Studies on Ergothioneine: Inhibitory effect on lipid peroxide formation in mouse liver, Chem. Pharm. Bull., 31 (5), 1983, pp. 1662-87. In studies comparing the inhibition of lipid peroxide (LPO) formation by various compounds in mouse liver, L-ergothioneine both inhibited LPO formation and enhanced the decomposition of existing LPO. Id. L-ergothioneine additionally has been shown to inhibit the damaging effects caused by the oxidation of iron-containing compounds, such as hemoglobin and myoglobin. These molecules are important in the body as carriers of oxygen, but because they contain divalent iron, they can interact with hydrogen peroxide via the Fenton reaction to produce the even more damaging hydroxyl radical. This is the mechanism by which damage occurs during so-called reperfusion injury. Because L-ergothioneine acts as a reducing agent of the ferryl-myoglobin molecule, it can protect tissues from reperfusion injury. Hanlon, D., Interaction of ergothioneine with metal ions and metalloenzymes, J. Med. Chem., 14 (11), 1971, pp. 1084-87. Although L-ergothioneine does not directly scavenge superoxide anion or hydrogen peroxide, it contributes to the control of these free radicals by participating in the activation of superoxide dismutase and glutathione peroxidase. Its protective effects on cell membranes and other organelles are of benefit in acute and chronic toxicity as well as in infectious diseases, because common pathogenic biomechanisms are active in both of these processes.

Ergothioneine in any form would be useful in the invention, including natural, semisynthetic, bioengineered, synthetic, extracted and combinations thereof and including any other active forms, such as racemic mixtures (D & L forms). L-ergothioneine is available commercially from Oxis International, Inc. or from dietary sources such as mushrooms.

### Ergothioneine or Ergothioneine Derivative

Ergothioneine or an ergothioneine derivative in the present invention can be either naturally derived or artificially synthesized using ordinary chemical and/or genetic engineering methods, and either of them can be included.

### Ergothioneine

Ergothioneine in the present invention implies any ergothioneine all or a part of which is known to be naturally derived or synthesized. This ergothioneine has cell preserving activity.

In the present invention, naturally derived ergothioneine is preferably obtained by a method described in U.S. Pat. No. 5,438,151, entitled, “Process for the Preparation of Ergothioneine”, the disclosure of which is herein incorporated by reference.

### Ergothioneine Derivative

L-ergothioneine is a phytonutrient and has been identified in mushrooms. It is a naturally occurring antioxidant that is very stable in the body. It is synthesized in fungi and microorganisms, and present in both plants and animals. Mammals and humans are unable to synthesize L-ergothioneine and must obtain it from dietary sources. It is readily absorbed and is active in most mammalian tissues, concentrating especially in the liver, where it prevents certain types of free-radical-induced damage to cell membranes and organelles. For example, exogenous L-ergothioneine has been shown to prevent lipid peroxidation by toxic compounds in the liver tissue of rats. In a recent study comparing the inhibition of lip peroxide (“LPO”) formation by various compounds in mouse liver, L-ergothioneine both inhibited LPO formation and enhanced the decomposition of existing LPO.

Additionally, L-ergothioneine serves as an antioxidant and a cellular protector against oxidative damage. The antioxidant properties of L-ergothioneine include: a scavenger of strong oxidants; chelation of various divalent metallic cations; and plays a key role in the oxidation of various hemoproteins. L-ergothioneine has been shown to inhibit the damaging effects caused by the oxidation of iron-containing compounds, such as hemoglobin and myoglobin. These molecules are important in the body as carriers of oxygen, but because they contain divalent iron, they can interact with hydrogen peroxide via the Fenton reaction to produce the even more damaging hydroxyl radical. This has been suggested as a mechanism by which damage occurs during so-called reperfusion injury.

Although L-ergothioneine does not directly scavenge superoxide anion or hydrogen peroxide, it contributes to the control of these free radicals by participating in the function of superoxide dismutase and glutathione peroxidase. Its protective effects on cell membranes and other organelles are of benefit in acute and chronic toxicity as well as in infectious diseases, because common pathogenic biomechanisms are active in both of these processes. Ergothioneine in any form would be useful in the invention, including natural, semisynthetic, bioengineered, synthetic, extracted and combinations thereof and including any other active forms, such as racemic mixtures (D & L forms). It is expected that daily microgram amounts of ergothioneine will be effective as an antioxidant. Other antioxidants, such as selenium, are known to be effective as antioxidants at these very low levels.

The expression “having cell preserving ability” herein means that cell preserving activity of a compound is recognized by those skilled in the art. For example, it means such a case that cell viability is improved when measured under the same conditions as described in the Example hereinafter.

Thus, the Ergothioneine derivative of the present invention may comprise an essential region only or may comprise at least the essential region and any nonessential region other than the essential region, as long as said Ergothioneine derivative has cell preserving ability.

### Naturally Derived Ergothioneine or Ergothioneine Derivative

According to one preferred embodiment of the present invention, the Ergothioneine and the Ergothioneine derivative can be derived from natural sources. Naturally derived products are advantageous because they are highly safe to the human body and relatively inexpensive. Such Ergothioneine or an Ergothioneine derivative can be used most appropriately as a medium supplement.

According to one preferred embodiment of the present invention, the Ergothioneine or the Ergothioneine derivative is extracted from mushrooms.

Further in the present invention, the Ergothioneine or the Ergothioneine derivative can be obtained from mushrooms by an ordinary extraction method. More specifically, for example, it may be obtained by methods described in U.S. Pat. No. 5,438,151, entitled, “Process for the Preparation of Ergothioneine”

### Ergothioneine or Ergothioneine Derivative Obtained by Chemical or Genetic Engineering Method

According to one preferred embodiment of the present invention, Ergothioneine and an Ergothioneine derivative can be artificially synthesized using an ordinary chemical or genetic engineering method. Typically, cell preserving ability of such Ergothioneine or an Ergothioneine derivative is equal to or higher than naturally derived one. Accordingly, such Ergothioneine or an Ergothioneine derivative can also be suitably used as a medium, or cell preserving supplement. Such chemical and genetic engineering methods for synthesis can be appropriately used in combination, if necessary.

In the present invention, the Ergothioneine and the Ergothioneine derivative can be produced by a genetic engineering method. Therefore, in the present invention, when mushroom cells may be preserved in tissue culture and the Ergothioneine or the Ergothioneine derivative produced may be harvested therefrom.

### Medium for Culturing, Preserving or Storing Cells

A cell culture medium according to the present invention comprises at least the abovementioned medium supplement for cell culture medium and a basal medium composition. Accordingly, if necessary, it can contain various cell growth factors, for example, binding proteins such as albumin and transferrin, hormones such as insulin, epithelial growth factor (EGF), fibroid cell growth factor and various steroid hormones, and cell adhesive factors such as fibronectin, as well as serum, as long as the abovementioned components are included.

According to a preferred embodiment of the present invention, the cell culture medium is preferably a medium which contains serum in a smaller amount than ordinary media, and more preferably a serum-free medium. The serum-free medium is a medium which contains no serum and may contain cell growth factors and hormones other than serum.

The amount of the Ergothioneine or the Ergothioneine derivative contained in the cell culture medium is not particularly limited, and can be appropriately changed depending on the kind of cells to be cultured, the purpose of the culture, the kind of the basal medium composition and the like.

According to a preferred embodiment of the present invention, the percentage of the Ergothioneine or the Ergothioneine derivative in the medium is 0.001 10% by weight, more preferably 0.02 0.5% by weight, and still more preferably 0.05 0.2% by weight.

The present invention exhibits a sufficient effect even when a small amount of the Ergothioneine or the Ergothioneine derivative is contained in the medium of the present invention. However, even if they are added in a large amount, there would be generally no substantial problem since Ergothioneine is nontoxic and highly water soluble.

When the medium supplement according to the present invention is advantageously used by adding it to an ordinary medium, it is desirable to dissolve the medium supplement in a small volume of the medium and then add it to the whole medium.

In the present invention, the basal medium composition comprises carbon sources assimilatable by general cells, digestible nitrogen sources and inorganic salts. More specifically, for example, inorganic salts, amino acids, glucose, and vitamins are included. If necessary, a trace substance for nutritional stimulation and an effective trace substance such as a precursor can be included in the basal medium composition.

Any medium composition known to the skilled in the art can be used as such a basal medium composition. More specifically, for example, MEM medium (H. Eagle, Science, 130, 432 (1959)), DMEM medium (R. Dulbecco, Virology, 8, 396 (1959)), RPMI 1640 medium (G. E. Moore, J.A.M.A., 199, 519 (1967)), Ham's F12 medium (R. G. Ham, Proc. Natl. Acad. Sci. U.S.A., 53, 288 (1965)), MCDB104 medium (W. L. Mckeehan, In Vitro, 13, 399 (1977)), and MCDB153 medium (D. M. Peehe, In Vitro, 16, 526 (1980)) can be used.

Other media which can be appropriately used in the present invention include serum-free medium ASF104 (Ajinomoto Co., Inc.), serum-free medium SF-02 (Sanko Junyaku Co., Ltd.), serum-free medium Hybridoma-SFM (Lifetech Oriental), serum-free medium BIO-MPM-1 (Biological Industries), serum-free medium EX-CELL™ 302-HDP (JRH Biosciences), serum-free medium Cosmedium 001 (Cosmo Bio), and serum-free medium SFM-101 (Nissui Pharmaceutical Co., Ltd.).

Cells which can be cultured in a medium of the present invention are not particularly limited and they can be either established cell lines or nonestablished normal cells obtained from biological tissues. Accordingly, cells of the present invention can be, for example, chondrocytes, red blood cells, stem cells, white blood cells, synoviocytes, plant cells, insect cells, bacterial cells and in a preferred embodiment, reproductive cells such as semen cells and oocytes as well as zygotes. Cells which could also be used with the present invention include cells which can produce proteins by themselves, cells which are transformed by genetic engineering to express heterologous proteins, or cells which are infected with various virus vectors.

Examples of the cells which can produce proteins by themselves include hybridoma cells producing monoclonal antibodies, leucocytes producing interferon (IFN)-α, fibroblasts producing IFN-β, lymphocytes producing IFN-γ, human kidney cells producing prourokinase (pro-UK) or UK, melanoma cells producing tissue plasminogen activator (tPA), In-111 cells producing insulin, HIT cells producing glucagon, HepG2 cells producing erythropoietin, and B151K12 cells producing interleukin-5.

Examples of the cell lines transformed by genetic engineering include Vero cells, HeLa cells, CHO (Chinese hamster ovary) cells, HKG cells, NIH3T3 cells, BHK cells, COS-1 cells, COS-7 cells, and myeloma cells.

Examples of the cells infected with virus vectors include those infected with retrovirus vectors, lentivirus vectors, adenovirus vectors, adeno-associated virus vectors, and herpesvirus vectors. These virus vectors can be genetically recombined by an ordinary genetic engineering method. Further, examples of the cells which are infected with these virus vectors and cultured using the medium of the present invention include HEK (human embryonic kidney) 293 cells, A549 cells, and PER.C6 cells.

Another preferred embodiment of the present invention provides a method of culturing cells, which comprises the steps of adding the medium supplement of the present invention to a cell culture medium and culturing cells using the resulting medium to grow the cells.

Culture conditions for this method, for example, the oxygen concentration, osmotic pressure, pH, temperature of the medium, can be appropriately changed depending on the kind of the cells to be cultured, the purpose of the culture, the volume of the culture, and the kind of the basal medium composition. Any culture system such as batch culture, continuous culture or perfusion culture can be used. High density culture can also be used.

Still another preferred embodiment of the present invention provides a method of producing a protein, comprising the steps of adding the medium supplement of the present invention to a cell culture medium, culturing cells capable of producing the protein using the resulting medium to grow the cells, and recovering the produced protein from said medium and/or said cells.

In the method of producing a protein according to the present invention, examples of the protein which can preferably be produced include monoclonal antibodies, IFN-α, IFN-β, INF-γ, pro-UK or UK, tPA, insulin, glucagon, erythropoietin, and interleukin-5.

The protein produced can be recovered using chemical or physical characteristics of the protein and isolated and purified by various ordinary isolation methods. For example, the protein can be recovered, isolated and purified by treatment with a protein coagulant, ultrafiltration, absorption chromatography, ion-exchange chromatography, affinity chromatography, molecular sieving chromatography, dialysis or the like, singly or in combination.

Another embodiment of the present invention provides a method of replicating a virus vector, which comprises the steps of adding the medium supplement of the present invention to a cell culture medium, culturing to grow cells infected with the virus vectors using the resulting medium and recovering the produced virus vectors from said medium and/or said cells.

Virus vectors replicable by the method of replication of the present invention are various virus vectors described above as examples and can be created by genetic recombination, if necessary.

Appropriately selected cells are infected with the virus vectors of interest by an ordinary method.

Further, the virus vectors can be recovered from grown cells by isolation and purification using various ordinary isolation methods such as ultrafiltration and centrifugation. Here it is desirable to appropriately select the method of recovering virus vectors according to the kind of virus vectors.

Generally, gene therapies are categorized into two kinds, i.e., ex vivo gene therapy and in vivo gene therapy. The former is a therapeutic method in which cells derived from a patient are first cultured outside the body and then treated for gene transfer, after which the cells are administered to the patient. The latter is a therapeutic method in which vectors with transferred genes are directly introduced into the patient's body.

The method according to the present invention can replicate virus vectors, into which genes used for such gene therapies are introduced, more efficiently than conventional methods. Further, the medium of the present invention exhibits excellent growth stimulating effect on the cells used for such a replication method, such as 293 cells.

### Experiment Setup

Table 1 below shows the 5 different extenders tested. The Stallion named IKE's semen was collected and centrifuged 1 to 1.5 in INRA® extender (IMV Technologies). The resulting sperm pellet was then split 5 ways and resuspended with the five different extenders tested. DMSO and EQUIPRO® cryoguards were frozen right away. INRA® was cooled to 5° C. before it was frozen. Semen was frozen using the ice cube freezer. Semen straws were thawed at 37° C. for 1 minute. Semen was diluted at one to ten in EQUIPRO® concentrate to analyze using sperm vision. All five extenders were measured for motility (total motility and forward progressive motility, FPM) using Sperm Vision® software, a high-resolution, rapid scan digital camera on a plain glass slide. Measurements were taken at 0 min post thaw, 30 min and 60 min post thaw.

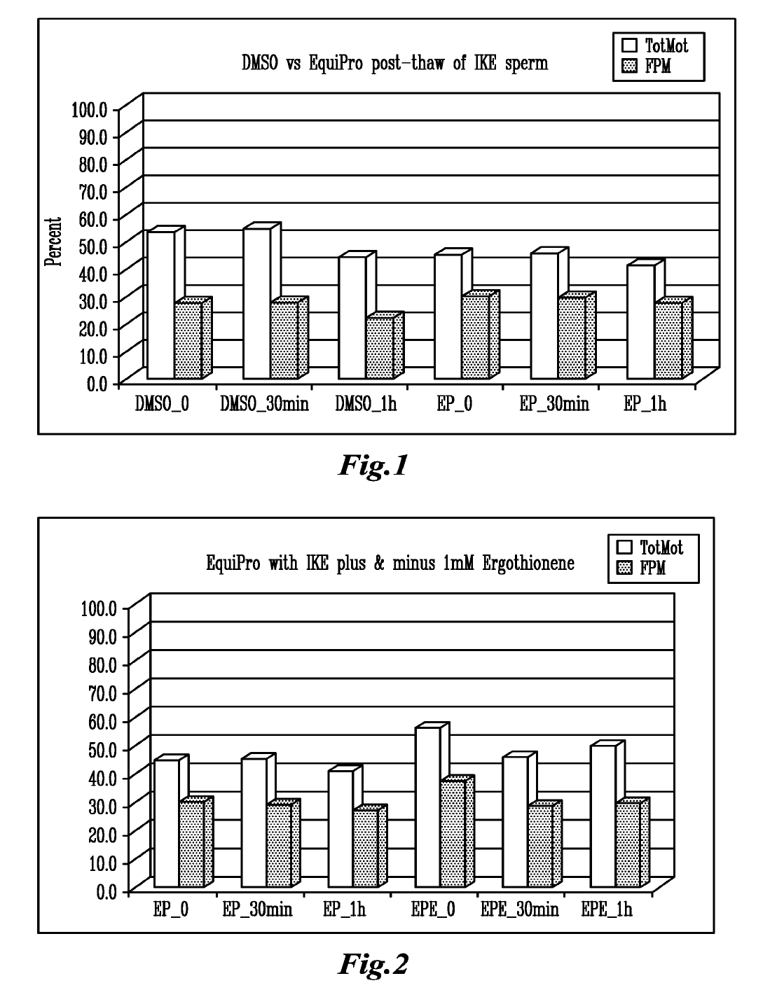

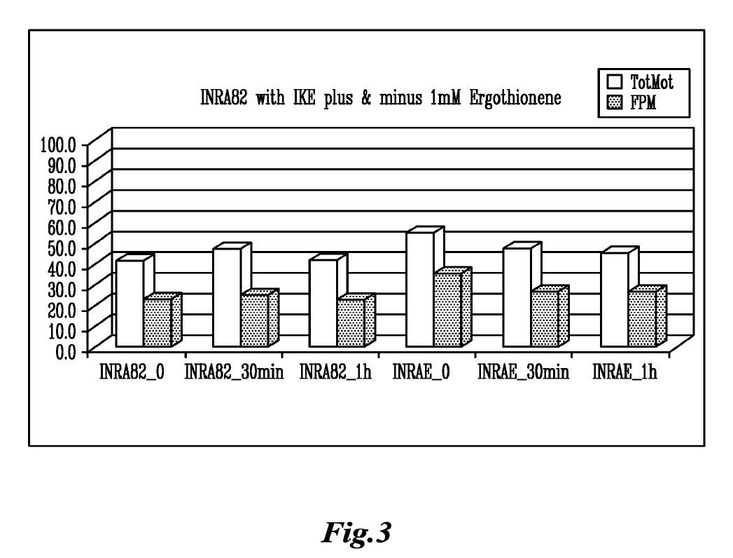

The results are depicted in Table 2 below and in FIGS. 1-3. Table 3 shows the raw data, and table 4 shows the raw data sorted.

TABLE 1

| EXTENDER | | ABBREV- |
|---|---|---|
| NO | DESCRIPTION | IATION |
| 1 | DMSO | DMSO |
| 2 | EQUIPRO CRYOGUARD COMPLETE | EP |
| 3 | EQUIPRO CRYOGUARD COMPLETE + | EPERG |
| | 1 MILLIMOLAR OF L-ERGOTHIONEINE | |
| 4 | INRA (FROM LISA METCALF) | INRA |
| 5 | INRA (FROM LISA METCALF) WITH 1 | INRAERG |
| | MILLIMOLAR OF L-ERGOTHIONEINE | |

TABLE 2

| Trmt | TotMot | FPM |
|---|---|---|
| DMSO_0 | 53.6 | 27.6 |
| DMSO_30min | 54.6 | 27.7 |
| DMSO_1h | 44.1 | 22.1 |
| EP_0 | 45.1 | 30.1 |
| EP_30min | 45.6 | 29.4 |
| EP_1h | 41.3 | 27.4 |
| EPE_0 | 56.5 | 37.9 |
| EPE_30min | 46.3 | 28.9 |
| EPE_1h | 50.2 | 29.8 |
| INRA82_0 | 41.3 | 22.8 |
| INRA82_30min | 47.2 | 25.1 |
| INRA82_0 | 41.7 | 22.9 |
| INRAE_0 | 54.9 | 35.4 |
| INRAE_30min | 47.7 | 26.7 |
| INRAE_1h | 45.5 | 26.6 |

TABLE 3

| | | | % | | | | | | | | | | | | |
|---|---|---|---|---|---|---|---|---|---|---|---|---|---|---|---|
| EXTENDER ID | STALLION | % Motile | Progressive | DAP | DCL | DSL | VAP | VCL | VSL | STR | LIN | WOB | ALH | BCF | Image |
| DMSO | IKE | 53.6 | 27.55 | 27.36 | 51.79 | 23.47 | 63.88 | 120.08 | 54.53 | 0.85 | 0.45 | 0.53 | 3.94 | 30.72 | −1 |
| DMSO | IKE | 46.62 | 24.32 | 32.93 | 60.86 | 29.78 | 73.16 | 134.99 | 66.1 | 0.9 | 0.48 | 0.54 | 3.49 | 33.76 | |
| DMSO | IKE | 59.89 | 32.81 | 27.9 | 52.14 | 22.64 | 65.83 | 123.16 | 52.65 | 0.79 | 0.42 | 0.53 | 4.35 | 30.17 | |
| DMSO | IKE | 48.46 | 25.76 | 28.59 | 56.09 | 24.76 | 68.26 | 132.68 | 59.28 | 0.86 | 0.44 | 0.51 | 3.81 | 35.05 | |
| DMSO | IKE | 57.06 | 32.06 | 26.8 | 50 | 22.83 | 63.83 | 117.38 | 54 | 0.84 | 0.46 | 0.54 | 4.05 | 28.73 | |
| DMSO | IKE | 58.02 | 26.54 | 24.44 | 46.17 | 21.5 | 58.04 | 107.84 | 51.35 | 0.88 | 0.47 | 0.53 | 3.9 | 29.4 | |
| DMSO | IKE | 51 | 24.16 | 28.03 | 53.09 | 24.73 | 64.52 | 121.87 | 56.58 | 0.87 | 0.46 | 0.52 | 3.75 | 29.99 | |
| DMSO | IKE | 51.44 | 24.63 | 22.91 | 45.05 | 18.98 | 51.83 | 101.3 | 42.63 | 0.82 | 0.42 | 0.51 | 3.84 | 29.07 | 0 |
| EP | IKE | 45.14 | 30.09 | 33.14 | 64.54 | 29.98 | 72.55 | 141.24 | 65.63 | 0.9 | 0.46 | 0.51 | 3.05 | 43 | −1 |
| EP | IKE | 37.87 | 22.72 | 31.85 | 59.58 | 28.93 | 68.06 | 126.94 | 61.88 | 0.9 | 0.48 | 0.53 | 2.8 | 46.22 | |
| EP | IKE | 33.33 | 22.22 | 34.47 | 63.08 | 31.98 | 75.35 | 137.91 | 69.91 | 0.92 | 0.5 | 0.54 | 2.69 | 47.11 | |
| EP | IKE | 36.23 | 23.18 | 31.15 | 65.93 | 28.54 | 72.47 | 151.9 | 66.67 | 0.92 | 0.43 | 0.47 | 2.78 | 43.71 | |
| EP | IKE | 45.45 | 29.87 | 34.78 | 67 | 31.1 | 76.62 | 148.32 | 68.37 | 0.89 | 0.46 | 0.51 | 3.38 | 40.74 | |
| EP | IKE | 50.72 | 37.68 | 34.42 | 67.62 | 31.41 | 73.88 | 145.7 | 67.47 | 0.91 | 0.46 | 0.5 | 3 | 43.14 | |
| EP | IKE | 55.55 | 34.56 | 29.98 | 58.45 | 26.75 | 66.15 | 128.86 | 58.93 | 0.89 | 0.45 | 0.51 | 3.26 | 42.01 | |
| EP | IKE | 54.92 | 39.43 | 34.84 | 68.42 | 31.19 | 75.23 | 147.15 | 67.32 | 0.89 | 0.45 | 0.51 | 3.09 | 41.24 | 0 |
| EPERG | IKE | 56.45 | 37.94 | 31.13 | 61.12 | 27.41 | 68.15 | 133.56 | 60.11 | 0.88 | 0.45 | 0.51 | 3.17 | 40.37 | −1 |
| EPERG | IKE | 51.69 | 34.74 | 31.78 | 62.48 | 28.26 | 68.82 | 135.22 | 61.14 | 0.88 | 0.45 | 0.5 | 2.87 | 41.97 | |
| EPERG | IKE | 57.04 | 35.21 | 30.13 | 59.39 | 26.83 | 67.9 | 133.8 | 60.6 | 0.89 | 0.45 | 0.5 | 3.09 | 42.51 | |
| EPERG | IKE | 58.26 | 39.37 | 34.34 | 68.18 | 30.84 | 73.91 | 146.82 | 66.39 | 0.89 | 0.45 | 0.5 | 3.03 | 44.79 | |
| EPERG | IKE | 48.81 | 33.85 | 31.24 | 62.71 | 26 | 66.85 | 133.76 | 55.79 | 0.83 | 0.41 | 0.49 | 3.36 | 38.44 | |
| EPERG | IKE | 54.62 | 39.49 | 29.12 | 56.03 | 25.9 | 64.65 | 124.21 | 57.56 | 0.89 | 0.46 | 0.52 | 3.5 | 37.6 | |
| EPERG | IKE | 53.7 | 37.03 | 30.81 | 59.71 | 26.39 | 67.21 | 129.49 | 57.87 | 0.86 | 0.44 | 0.51 | 3.16 | 37.79 | |
| EPERG | IKE | 69.4 | 45.52 | 30.57 | 59.54 | 27.31 | 67.42 | 131.1 | 60.33 | 0.89 | 0.46 | 0.51 | 3.15 | 39.1 | 0 |
| INRA | IKE | 41.34 | 22.76 | 29.3 | 56.35 | 26.24 | 66.38 | 126.81 | 59.45 | 0.89 | 0.46 | 0.52 | 3.49 | 37.42 | −1 |
| INRA | IKE | 38.88 | 19.75 | 29.38 | 52.65 | 26.39 | 68.88 | 122.81 | 62.19 | 0.9 | 0.5 | 0.56 | 3.5 | 37.06 | |
| INRA | IKE | 44.17 | 25.76 | 31.04 | 57.43 | 28.05 | 70.02 | 128.74 | 63.03 | 0.9 | 0.48 | 0.54 | 3.45 | 40.08 | |
| INRA | IKE | 44.02 | 25 | 29.92 | 56.85 | 26.88 | 68.1 | 128.28 | 61.07 | 0.89 | 0.47 | 0.53 | 3.45 | 36.31 | |
| INRA | IKE | 47.61 | 25.39 | 29.95 | 58.8 | 27.1 | 67.47 | 132.03 | 61.15 | 0.9 | 0.46 | 0.51 | 3.47 | 39.17 | |
| INRA | IKE | 47.33 | 25.44 | 26.76 | 53.97 | 23.63 | 60.36 | 120.77 | 53.02 | 0.87 | 0.43 | 0.49 | 3.45 | 34.91 | |
| INRA | IKE | 28.38 | 15.48 | 30.38 | 59.52 | 27.43 | 69.94 | 135 | 63.38 | 0.9 | 0.46 | 0.51 | 3.69 | 33.72 | |
| INRA | IKE | 36.42 | 21.19 | 27.62 | 55.07 | 24.11 | 60.4 | 120.34 | 52.87 | 0.87 | 0.43 | 0.5 | 3.5 | 39.43 | 0 |
| INRAERG | IKE | 54.9 | 35.38 | 30.44 | 58.71 | 26.99 | 67.88 | 130.48 | 60.17 | 0.88 | 0.46 | 0.52 | 3.29 | 37.98 | −1 |
| INRAERG | IKE | 56.29 | 38.51 | 30.26 | 56.52 | 27.21 | 65.99 | 122.68 | 59.42 | 0.9 | 0.48 | 0.53 | 2.8 | 39.84 | |
| INRAERG | IKE | 61.65 | 39.84 | 31.18 | 59.47 | 28.05 | 70.15 | 133.42 | 63.08 | 0.89 | 0.47 | 0.52 | 3.34 | 36.53 | |
| INRAERG | IKE | 52.73 | 36.98 | 30.82 | 60.73 | 26.32 | 69 | 135.64 | 58.91 | 0.85 | 0.43 | 0.5 | 3.34 | 39.69 | |
| INRAERG | IKE | 58.2 | 37.31 | 26.74 | 51.01 | 23.71 | 61.29 | 116.42 | 54.41 | 0.88 | 0.46 | 0.52 | 3.66 | 35.32 | |
| INRAERG | IKE | 52.45 | 29.5 | 30.71 | 59.09 | 27.55 | 68.84 | 132.37 | 61.66 | 0.89 | 0.46 | 0.52 | 3.22 | 39.18 | |
| INRAERG | IKE | 56.16 | 30.82 | 30.45 | 61.46 | 26.52 | 67.89 | 136.53 | 59.14 | 0.87 | 0.43 | 0.49 | 3.39 | 36.6 | |
| INRAERG | IKE | 45.04 | 34.23 | 33.69 | 64.3 | 30.48 | 73.46 | 139.29 | 66.37 | 0.9 | 0.47 | 0.52 | 3.23 | 39 | 0 |
| DMSO | IKE | 54.59 | 27.68 | 27.64 | 52.66 | 23.85 | 63.1 | 119.86 | 54.57 | 0.86 | 0.45 | 0.52 | 3.76 | 33.59 | |
| DMSO | IKE | 55.55 | 23.93 | 27.21 | 49.64 | 23.6 | 64.58 | 117.68 | 56.54 | 0.87 | 0.48 | 0.54 | 3.19 | 34.76 | |
| DMSO | IKE | 59.6 | 23.17 | 32.32 | 59.83 | 29.15 | 73.28 | 134.94 | 65.99 | 0.9 | 0.48 | 0.54 | 3.81 | 36.99 | |
| DMSO | IKE | 48.38 | 22.58 | 28.07 | 57.33 | 23.77 | 61.85 | 124.93 | 52.45 | 0.84 | 0.41 | 0.49 | 3.67 | 32.69 | |
| DMSO | IKE | 49.09 | 26.36 | 28.28 | 54.22 | 24.28 | 64.62 | 124.07 | 55.38 | 0.85 | 0.44 | 0.52 | 3.68 | 36.7 | |
| DMSO | IKE | 53.98 | 30.97 | 29.09 | 55.12 | 25.43 | 64.91 | 122.75 | 56.87 | 0.87 | 0.46 | 0.52 | 3.58 | 33.35 | |
| DMSO | IKE | 62.31 | 34.78 | 26.04 | 51.78 | 21.95 | 60.15 | 119.84 | 50.85 | 0.84 | 0.42 | 0.5 | 4.52 | 32.84 | |
| DMSO | IKE | 52.3 | 33.07 | 23.91 | 42.89 | 20.3 | 55.66 | 99.69 | 47.46 | 0.85 | 0.47 | 0.55 | 3.51 | 29.73 | |
| EP | IKE | 45.61 | 29.38 | 29.33 | 59.94 | 26.02 | 64.32 | 131.44 | 56.92 | 0.88 | 0.43 | 0.48 | 2.74 | 41.88 | |
| EP | IKE | 60.86 | 47.82 | 36.81 | 70.14 | 34.37 | 79.97 | 153.22 | 74.73 | 0.93 | 0.48 | 0.52 | 2.52 | 46.66 | |
| EP | IKE | 41.81 | 27.27 | 30.9 | 60.44 | 27.18 | 64.14 | 125.58 | 56.43 | 0.87 | 0.44 | 0.51 | 2.62 | 43.89 | |
| EP | IKE | 47.27 | 32.72 | 26.96 | 54.6 | 23.6 | 61.58 | 125.32 | 53.31 | 0.86 | 0.42 | 0.49 | 2.47 | 45.01 | |
| EP | IKE | 41.17 | 29.41 | 28.49 | 58.82 | 25.9 | 63.3 | 129.04 | 57.61 | 0.9 | 0.44 | 0.49 | 2.7 | 45.6 | |
| EP | IKE | 36.9 | 20.23 | 26.67 | 54.23 | 23.82 | 57.15 | 116.95 | 50.9 | 0.89 | 0.43 | 0.48 | 2.33 | 42.8 | |
| EP | IKE | 45.34 | 27.9 | 26.15 | 57.84 | 21.37 | 60.87 | 133.5 | 49.55 | 0.81 | 0.37 | 0.45 | 3.58 | 32.46 | |
| EP | IKE | 53.22 | 29.03 | 28.92 | 61.8 | 25.71 | 60.61 | 129.4 | 53.94 | 0.88 | 0.41 | 0.46 | 2.72 | 38.77 | |
| EPERG | IKE | 46.34 | 28.92 | 28.19 | 54.87 | 24.98 | 61.48 | 119.53 | 54.49 | 0.88 | 0.45 | 0.51 | 2.88 | 40.08 | |
| EPERG | IKE | 49.03 | 27.88 | 30.34 | 54.94 | 27.66 | 65.16 | 117.94 | 59.48 | 0.91 | 0.5 | 0.55 | 2.8 | 45.09 | |
| EPERG | IKE | 48.71 | 32.47 | 29.02 | 54.44 | 26.19 | 63.81 | 119.32 | 57.51 | 0.9 | 0.48 | 0.53 | 2.84 | 40.72 | |
| EPERG | IKE | 37.6 | 25.64 | 29.23 | 60.12 | 26.08 | 63.69 | 131.26 | 56.77 | 0.89 | 0.43 | 0.48 | 3.12 | 41.36 | |
| EPERG | IKE | 50 | 27.27 | 26.7 | 55.11 | 23.3 | 57.31 | 117.84 | 50.06 | 0.87 | 0.42 | 0.48 | 2.8 | 40.7 | |
| EPERG | IKE | 46.15 | 28.84 | 28.8 | 55.61 | 24.75 | 62.67 | 121.09 | 53.79 | 0.85 | 0.44 | 0.51 | 2.98 | 37.11 | |
| EPERG | IKE | 47.25 | 31.86 | 24.81 | 48.92 | 21.57 | 55.58 | 109.4 | 48.45 | 0.87 | 0.44 | 0.5 | 2.77 | 35.35 | |
| INRA | IKE | 47.22 | 25.12 | 26.29 | 51.04 | 23.19 | 59.02 | 113.52 | 52.11 | 0.88 | 0.45 | 0.51 | 3.03 | 37.74 | |
| INRA | IKE | 46.51 | 27.9 | 28.16 | 50.97 | 25.04 | 66.53 | 118.98 | 59.15 | 0.88 | 0.49 | 0.55 | 2.9 | 37.21 | |
| INRA | IKE | 54.23 | 31.63 | 27.96 | 52.59 | 25.26 | 64.45 | 120 | 58.41 | 0.9 | 0.48 | 0.53 | 3.09 | 42.3 | |
| INRA | IKE | 51.39 | 27.93 | 24.67 | 44.75 | 22.01 | 55.7 | 100.31 | 49.76 | 0.89 | 0.49 | 0.55 | 3.13 | 36.19 | |
| INRA | IKE | 51.21 | 21.13 | 25.14 | 53.45 | 21.01 | 54.17 | 114.44 | 45.39 | 0.83 | 0.39 | 0.47 | 3.37 | 34.94 | |
| INRA | IKE | 36.84 | 15.78 | 24.19 | 49.74 | 20.88 | 50.9 | 104.77 | 43.97 | 0.86 | 0.41 | 0.48 | 2.46 | 36.55 | |
| INRA | IKE | 41.54 | 19.71 | 27.15 | 55.42 | 23.77 | 57.26 | 116.79 | 50.17 | 0.87 | 0.42 | 0.49 | 3.19 | 36.52 | |
| INRA | IKE | 44.16 | 26.66 | 24.47 | 53.19 | 21.21 | 53.44 | 115.95 | 45.89 | 0.85 | 0.39 | 0.46 | 2.84 | 37.01 | |
| INRAERG | IKE | 47.71 | 26.65 | 24.66 | 48.56 | 21.27 | 53.8 | 105.49 | 46.39 | 0.86 | 0.43 | 0.51 | 2.97 | 34.3 | |
| INRAERG | IKE | 47.22 | 33.33 | 22.85 | 41.16 | 20.01 | 48.83 | 88.45 | 42.84 | 0.87 | 0.48 | 0.55 | 2.55 | 31.96 | |
| INRAERG | IKE | 50 | 16.66 | 25.49 | 55.23 | 22.07 | 56.84 | 122.49 | 49.57 | 0.87 | 0.4 | 0.46 | 3.01 | 40.28 | |
| INRAERG | IKE | 46.26 | 23.88 | 30.85 | 61.36 | 26.73 | 64.23 | 127.58 | 55.64 | 0.86 | 0.43 | 0.5 | 2.68 | 43.89 | |
| INRAERG | IKE | 48.86 | 25 | 26.01 | 51.47 | 22.98 | 54.59 | 107.46 | 48.22 | 0.88 | 0.44 | 0.5 | 3.09 | 36.46 | |
| INRAERG | IKE | 44.85 | 28.03 | 24.48 | 51.47 | 20.74 | 54.57 | 113.79 | 46.47 | 0.85 | 0.4 | 0.47 | 2.91 | 34.33 | |
| INRAERG | IKE | 50.41 | 28.92 | 22.72 | 44.06 | 19.3 | 51.07 | 97.67 | 43.35 | 0.84 | 0.44 | 0.52 | 3.16 | 32.08 | |
| INRAERG | IKE | 46.36 | 26.36 | 24.63 | 47.89 | 21.07 | 54.78 | 106.2 | 46.49 | 0.84 | 0.43 | 0.51 | 3.36 | 30.24 | |
| DMSO | IKE | 44.12 | 22.12 | 30.03 | 57.34 | 26.75 | 66.38 | 126.29 | 59.08 | 0.89 | 0.46 | 0.52 | 3.54 | 34.66 | |
| DMSO | IKE | 42.85 | 21.9 | 28.26 | 50.07 | 25.36 | 58.64 | 103.89 | 52.62 | 0.89 | 0.5 | 0.56 | 2.7 | 35.58 | |
| DMSO | IKE | 41.72 | 22.3 | 31.16 | 58.01 | 28.55 | 69.48 | 128.38 | 63.55 | 0.91 | 0.49 | 0.54 | 3.65 | 36.84 | |
| DMSO | IKE | 41.59 | 19.46 | 33.09 | 66.96 | 29.58 | 74.02 | 149.23 | 66.28 | 0.89 | 0.44 | 0.49 | 3.47 | 34.58 | |
| DMSO | IKE | 39.16 | 20.27 | 30.67 | 61.82 | 26.93 | 68.12 | 136.84 | 59.93 | 0.87 | 0.43 | 0.49 | 3.75 | 34.79 | |
| DMSO | IKE | 48.83 | 32.55 | 28.68 | 52.56 | 24.99 | 65.92 | 120.52 | 57.32 | 0.86 | 0.47 | 0.54 | 3.83 | 32.02 | |
| DMSO | IKE | 50.83 | 23.33 | 28.46 | 53.23 | 25.41 | 63.42 | 117.75 | 56.39 | 0.88 | 0.47 | 0.53 | 3.47 | 33.41 | |
| DMSO | IKE | 46.8 | 17.02 | 30.15 | 60.72 | 26.51 | 63.87 | 128.78 | 56.1 | 0.87 | 0.43 | 0.49 | 3.83 | 35.81 | |
| EP | IKE | 41.26 | 27.43 | 27.41 | 57.37 | 24.57 | 60.03 | 125.59 | 53.86 | 0.89 | 0.42 | 0.47 | 2.87 | 41.09 | |
| EP | IKE | 48.52 | 30.88 | 29.29 | 58.67 | 26.48 | 64.19 | 128.77 | 58.04 | 0.9 | 0.45 | 0.49 | 2.83 | 43.99 | |
| EP | IKE | 41.66 | 27.08 | 26.27 | 56.34 | 23.79 | 59.86 | 129.04 | 54.23 | 0.9 | 0.42 | 0.46 | 2.99 | 41.56 | |
| EP | IKE | 26.86 | 16.41 | 30.04 | 64.79 | 26.98 | 62.39 | 134.5 | 56.05 | 0.89 | 0.41 | 0.46 | 2.82 | 43.4 | |
| EP | IKE | 37.14 | 18.57 | 24.99 | 50.61 | 21.94 | 53.35 | 106.96 | 46.94 | 0.87 | 0.43 | 0.49 | 2.93 | 38.56 | |
| EP | IKE | 44.77 | 34.32 | 26.86 | 51.04 | 24.75 | 58.78 | 111.52 | 54.2 | 0.92 | 0.48 | 0.52 | 2.62 | 41.24 | |
| EP | IKE | 52.38 | 33.33 | 26.68 | 58.33 | 23.18 | 58.94 | 128.01 | 51.36 | 0.87 | 0.4 | 0.46 | 2.78 | 39.84 | |
| EP | IKE | 37.7 | 32.78 | 28.42 | 63.96 | 25.2 | 61.54 | 138.61 | 54.55 | 0.88 | 0.39 | 0.44 | 3.12 | 38.94 | |
| EPERG | IKE | 50.24 | 29.8 | 27.72 | 57.41 | 24.04 | 60.09 | 124.36 | 52.08 | 0.86 | 0.41 | 0.48 | 3.02 | 38.77 | |
| EPERG | IKE | 48.92 | 32.37 | 26.13 | 51.83 | 22.81 | 57.12 | 113.71 | 49.87 | 0.87 | 0.43 | 0.5 | 2.75 | 35.96 | |
| EPERG | IKE | 48.38 | 32.25 | 31.07 | 63.68 | 27.72 | 68.15 | 139.66 | 60.6 | 0.88 | 0.43 | 0.48 | 3.21 | 43.2 | |
| EPERG | IKE | 55.83 | 33.33 | 27.71 | 55.56 | 24.15 | 60.07 | 120.22 | 52.29 | 0.87 | 0.43 | 0.49 | 3.06 | 39.91 | |
| EPERG | IKE | 61.32 | 38.67 | 30.27 | 63.41 | 25.67 | 65.09 | 136.44 | 55.03 | 0.84 | 0.4 | 0.47 | 3.12 | 40.51 | |
| EPERG | IKE | 51.77 | 24.82 | 25.31 | 54.96 | 21.96 | 55.88 | 120.89 | 48.7 | 0.87 | 0.4 | 0.46 | 2.89 | 38.64 | |
| EPERG | IKE | 45.04 | 24.32 | 26.42 | 55.91 | 22.39 | 55.76 | 117.45 | 47.28 | 0.84 | 0.4 | 0.47 | 3.26 | 33.35 | |
| EPERG | IKE | 35.21 | 19.71 | 24.3 | 54.19 | 20.79 | 50.83 | 113.3 | 43.39 | 0.85 | 0.38 | 0.44 | 2.83 | 37.57 | |
| INRA | IKE | 41.7 | 22.89 | 26.02 | 50.96 | 22.57 | 58.16 | 112.72 | 50.38 | 0.86 | 0.44 | 0.51 | 3.39 | 34.22 | |
| INRA | IKE | 44.18 | 27.9 | 33.29 | 62.4 | 30.68 | 72.41 | 134.17 | 66.77 | 0.92 | 0.49 | 0.53 | 3.49 | 40.06 | |
| INRA | IKE | 48.38 | 27.09 | 25.05 | 46.15 | 21.9 | 58.57 | 107.6 | 51.09 | 0.87 | 0.47 | 0.54 | 3.34 | 34.76 | |
| INRA | IKE | 40 | 22.22 | 25.05 | 54.04 | 21.89 | 56.76 | 120.97 | 49.3 | 0.86 | 0.4 | 0.46 | 3.23 | 34.27 | |
| INRA | IKE | 41.02 | 21.15 | 23.2 | 47.39 | 19.89 | 51.99 | 104.79 | 44.39 | 0.85 | 0.42 | 0.49 | 3.38 | 31.21 | |
| INRA | IKE | 43.33 | 21.66 | 23.08 | 43.36 | 18.64 | 52.21 | 96.67 | 42.33 | 0.81 | 0.43 | 0.54 | 3.47 | 31.6 | |
| INRA | IKE | 37.83 | 19.59 | 27.6 | 53.28 | 23.27 | 59.28 | 113.89 | 50.13 | 0.84 | 0.44 | 0.52 | 3.73 | 34.02 | |
| INRA | IKE | 35.93 | 21.09 | 24.92 | 52.6 | 21.75 | 54.96 | 114.52 | 47.89 | 0.87 | 0.41 | 0.47 | 3.01 | 33.23 | |
| INRAERG | IKE | 45.51 | 26.59 | 26.28 | 52.72 | 22.89 | 57.26 | 114.21 | 49.92 | 0.87 | 0.43 | 0.5 | 3.13 | 38.26 | |
| INRAERG | IKE | 54.44 | 27.77 | 32.82 | 61.36 | 29.78 | 69.29 | 129.24 | 62.87 | 0.9 | 0.48 | 0.53 | 3.06 | 43.71 | |
| INRAERG | IKE | 45.79 | 34.57 | 24.3 | 42.22 | 20.68 | 54.12 | 93.22 | 46.13 | 0.85 | 0.49 | 0.58 | 3.42 | 32.75 | |
| INRAERG | IKE | 63.63 | 37.87 | 24.57 | 51.16 | 20.88 | 53.37 | 111.55 | 45.28 | 0.84 | 0.4 | 0.47 | 3.2 | 40.99 | |
| INRAERG | IKE | 46.05 | 19.73 | 26.87 | 55.34 | 24.45 | 61.76 | 124.65 | 56.25 | 0.91 | 0.45 | 0.49 | 2.66 | 41.95 | |
| INRAERG | IKE | 38.2 | 23.59 | 23.35 | 52.23 | 20.19 | 49.11 | 109.61 | 42.46 | 0.86 | 0.38 | 0.44 | 3.1 | 37.38 | |
| INRAERG | IKE | 35.71 | 18.36 | 22.77 | 51.05 | 19.92 | 50.35 | 111.93 | 44.25 | 0.87 | 0.39 | 0.44 | 2.74 | 37.91 | |
| INRAERG | IKE | 40.22 | 25.28 | 29.38 | 62.39 | 25 | 63.68 | 134.59 | 54.27 | 0.85 | 0.4 | 0.47 | 3.3 | 36.86 | |

TABLE 4

| | | | % | | | | | | | | | | | | |
|---|---|---|---|---|---|---|---|---|---|---|---|---|---|---|---|
| EXTENDER ID | STALLION | % Motile | Progressive | DAP | DCL | DSL | VAP | VCL | VSL | STR | LIN | WOB | ALH | BCF | Image |
| DMSO | IKE | 53.6 | 27.55 | 27.36 | 51.79 | 23.47 | 63.88 | 120.08 | 54.53 | 0.85 | 0.45 | 0.53 | 3.94 | 30.72 | −1 |
| DMSO | IKE | 54.59 | 27.68 | 27.64 | 52.66 | 23.85 | 63.1 | 119.86 | 54.57 | 0.86 | 0.45 | 0.52 | 3.76 | 33.59 | |
| DMSO | IKE | 44.12 | 22.12 | 30.03 | 57.34 | 26.75 | 66.38 | 126.29 | 59.08 | 0.89 | 0.46 | 0.52 | 3.54 | 34.66 | |
| EP | IKE | 45.14 | 30.09 | 33.14 | 64.54 | 29.98 | 72.55 | 141.24 | 65.63 | 0.9 | 0.46 | 0.51 | 3.05 | 43 | −1 |
| EP | IKE | 45.61 | 29.38 | 29.33 | 59.94 | 26.02 | 64.32 | 131.44 | 56.92 | 0.88 | 0.43 | 0.48 | 2.74 | 41.88 | |
| EP | IKE | 41.26 | 27.43 | 27.41 | 57.37 | 24.57 | 60.03 | 125.59 | 53.86 | 0.89 | 0.42 | 0.47 | 2.87 | 41.09 | |
| EPERG | IKE | 56.45 | 37.94 | 31.13 | 61.12 | 27.41 | 68.15 | 133.56 | 60.11 | 0.88 | 0.45 | 0.51 | 3.17 | 40.37 | −1 |
| EPERG | IKE | 46.34 | 28.92 | 28.19 | 54.87 | 24.98 | 61.48 | 119.53 | 54.49 | 0.88 | 0.45 | 0.51 | 2.88 | 40.08 | |
| EPERG | IKE | 50.24 | 29.8 | 27.72 | 57.41 | 24.04 | 60.09 | 124.36 | 52.08 | 0.86 | 0.41 | 0.48 | 3.02 | 38.77 | |
| INRA | IKE | 41.34 | 22.76 | 29.3 | 56.35 | 26.24 | 66.38 | 126.81 | 59.45 | 0.89 | 0.46 | 0.52 | 3.49 | 37.42 | −1 |
| INRA | IKE | 47.22 | 25.12 | 26.29 | 51.04 | 23.19 | 59.02 | 113.52 | 52.11 | 0.88 | 0.45 | 0.51 | 3.03 | 37.74 | |
| INRA | IKE | 41.7 | 22.89 | 26.02 | 50.96 | 22.57 | 58.16 | 112.72 | 50.38 | 0.86 | 0.44 | 0.51 | 3.39 | 34.22 | |
| INRAERG | IKE | 54.9 | 35.38 | 30.44 | 58.71 | 26.99 | 67.88 | 130.48 | 60.17 | 0.88 | 0.46 | 0.52 | 3.29 | 37.98 | −1 |
| INRAERG | IKE | 47.71 | 26.65 | 24.66 | 48.56 | 21.27 | 53.8 | 105.49 | 46.39 | 0.86 | 0.43 | 0.51 | 2.97 | 34.3 | |
| INRAERG | IKE | 45.51 | 26.59 | 26.28 | 52.72 | 22.89 | 57.26 | 114.21 | 49.92 | 0.87 | 0.43 | 0.5 | 3.13 | 38.26 | |

1. A method of preserving cells or components thereof comprising: adding to said cells an effective amount of ergothioneine.

2. The method of claim 1 wherein said ergothioneine is L-ergothioneine.

3. The method of claim 1 wherein said ergothioineine is purified from mushrooms.

4. The method of claim 1 wherein said ergothioineine is recombinant.

5. The method of claim 1 wherein said cells are in cell culture.

6. The method of claim 1 wherein said cells are selected from the group consisting of: chondrocytes, red blood cells, stem cells, white blood cells, synoviocytes, plant cells, insect cells, bacterial cells, semen cells, oocytes, embryonic stem cells and zygotes.

7. The method of claim 1 wherein said cells are semen cells.

8. The method of claim 7 wherein said semen cells are reconstituted from frozen cells.

9. The method of claim 7 wherein said ergothioneine is added before freezing said cells.

10. (canceled)

11. A cell preservative composition for cell culture, storage and preservation comprising: ergothioneine and a carrier.

12. The preservative composition of claim 11 wherein said preservative composition includes a cell culture medium that has been supplemented with ergothioneine.

13. The preservative composition of claim 11 wherein said preservative composition includes a semen extender that has been supplemented with ergothioneine.

14. A supplement for enhancing the viability of cells, tissues or components thereof in storage, culture or suspension comprising: an effective amount of ergothioneine and a carrier.

15. The supplement of claim 14 wherein said supplement is added to a semen extender.

16. The supplement of claim 15 wherein said supplement is added to a culture medium.

17. The supplement of claim 15 wherein said supplement is added to cells before lyophilization.

18. The supplement of claim 15 wherein said supplement is added to cells upon reconstitution.

19. A method for preserving sperm cells comprising:
adding to said cells an effective amount of ergothioneine.

20. The method of claim 19 wherein said ergothioneine is added in an amount of 1 mM.

21. The method of claim 19 wherein said ergothioneine is L-ergothioneine.

22. The method of claim 19 wherein said sperm cells are from an animal selected from the group consisting of:
equine, porcine, avian, human, bovine or canine

23. The method of claim 19 wherein said sperm cells are equine.

24. The method of claim 19 further comprising the step of:
Adding semen extender to said sperm cells.

25. The method of claim 19 wherein said cells are lyophilized.

26. A collection of preserved sperm cells made by the method of claim 19.
